(19) United States
(12) Patent Application Publication
(10) Pub. No.: US 2017/0234887 A1
(43) Pub. Date: Aug. 17, 2017

(54) METHOD FOR DETECTING A SPATIAL PROXIMITY OF A FIRST AND A SECOND EPITOPE

(71) Applicant: KONINKLIJKE PHILIPS N.V., EINDHOVEN (NL)

(72) Inventors: FREEK VAN HEMERT, EINDHOVEN (NL); REINHOLD WIMBERGER-FRIEDL, EINDHOVEN (NL); DIANNE ARNOLDINA MARGARETHA WILHELMINA VAN STRIJP, EINDHOVEN (NL)

(21) Appl. No.: 15/515,329

(22) PCT Filed: Sep. 30, 2015
(86) PCT No.: PCT/EP2015/072495

§ 371 Date: Mar. 29, 2017

(30) Foreign Application Priority Data
Sep. 30, 2014 (EP) 14187001.4

(51) Int. Cl. G01N 33/68; G01N 33/542; C12Q 1/68

(52) U.S. Cl. CPC G01N 33/6878; C12Q 1/6804; G01N 33/542; G01N 33/6893; G01N 2458/10; G01N 2800/7057; G01N 2800/52; G01N 2800/56

(57) ABSTRACT

The present invention relates to a method for detecting a spatial proximity of a first and a second epitope (11, 21) of a protein or of a first and a second protein (10, 20) of a protein complex (1) in a sample of a subject. The method comprises binding a first binding member (30) having a first oligonucleotide (31) conjugated thereto to the first epitope (11), binding a second binding member (40) having a second oligonucleotide (41) conjugated thereto to the second epitope (21), and determining whether a Fluorescence Resonance Energy Transfer (FRET) effect is present between a donor fluorophore (32) and an acceptor fluorophore (42), which are associated with the first oligonucleotide (31) and the second oligonucleotide (41), wherein the presence of the FRET effect indicates a spatial proximity of the first and the second oligonucleotide (31, 41) and, thus, the spatial proximity of the first and the second epitope (11, 21).

### FIELD OF THE INVENTION

The present invention relates to a method for detecting a spatial proximity of a first and a second epitope of a protein or of a first and a second protein of a protein complex in a sample of a subject. The present invention further relates to a method for stratification of a subject suffering from a disease for assessing the suitability of a therapy and/or for prognosis of the outcome of a disease of a subject and/or for prediction and/or detection of therapy resistance of a subject suffering from a disease towards a therapy. Furthermore, the present invention relates to a novel kit and corresponding uses thereof.

### BACKGROUND OF THE INVENTION

Proximity Ligation Assay (PLA) is a method that is capable of reporting the co-location of two proteins (see Weibrecht I. et al., “Proximity ligation assays: A recent addition to the proteomics toolbox”, Expert Review of Proteomics, Vol. 7, No. 3, 2010, pages 401 to 409). The method uses two antibodies each labeled with a single-stranded DNA oligonucleotide. The oligonucleotides are both needed to form a closed circle out of two secondary DNA oligonucleotides, which are added to the sample after the antibodies have bound to their epitopes. Once the circle is formed, Rolling Circle amplification (RCA) is used to create hundreds of copies of the circular template. Finally, complementary probes, labeled with a fluorophore, are annealed to the RCA product yielding a bright spot at the place where the two proteins are co-located.

The standard PLA protocol (even with labeled primary antibodies) takes about 6.5 hours to complete. The longest step in which RCA takes place takes about 1 hour and 40 minutes and requires an enzyme. Apart from the long time this reaction takes, the usage of an enzyme makes the entire assay difficult to integrate into a device, mostly because the stable storage of sensitive enzymes is generally a problem. It would therefore be desirable to have a technology for determining a spatial proximity or co-location of two proteins by in-situ staining on tissue and/or cells (cell agglomerates) and/or a body fluid of a patient that either does not require the use of an enzyme or that, at least, does not require complex enzymatic reactions, such as sequence amplification or the like.

WO 2005/059509 A3 discloses compositions and methods that are useful in the identification and quantification of any polypeptide or macromolecular complex using a set of co-aptamer constructs. Aptamer constructs are constructed that bind to unique epitopes of a polypeptide of macromolecular construct. Those aptamer constructs contain an epitope binding site, a co-aptamer binding site, and a detectable label. In the presence of the cognate polypeptide, analyte-polypeptide complex, or other macromolecular complex, the co-aptamers associate with one another to produce a detectable signal. The co-aptamer constructs may be joined by a linker to produce a bivalent aptamer construct.

WO 2012/152942 A1 relates to a proximity-probe based detection assay for detecting an analyte in a sample and in particular to a method that comprises the use of at least one set of at least first and second proximity probes, which probes each comprise an analyte-binding domain and a nucleic acid domain and can simultaneously bind to the analyte directly or indirectly, wherein the nucleic acid domain of at least one of said proximity probes comprises a hairpin structure that can be unfolded by cleavage of the nucleic acid domain to generate at least one ligatable free end or region of complementarity to another nucleic acid molecule in said sample, wherein when the probes bind to said analyte unfolding said hairpin structure allows the nucleic acid domains of said at least first and second proximity probes to interact directly or indirectly.

WO 2010/006291 A1 provides an approach for the determination of the activation states of a plurality of proteins in single cells. This approach permits the rapid detection of heterogeneity in a complex cell population based on activation states, expression markers and other criteria, and the identification of cellular subsets that exhibit correlated changes in activation within the cell population. Moreover, this approach allows the correlation of cellular activities or properties. In addition, the use of modulators of cellular activation allows for characterization of pathways and cell populations. Several exemplary diseases that can be analyzed using the approach include AML, MDS, and MPN.

### SUMMARY OF THE INVENTION

It is an object of the present invention to provide a method for detecting a spatial proximity of a first and a second epitope of a protein or of a first and a second protein of a protein complex in a sample of a subject, wherein the method solves, or at least reduces the aforementioned problem of the PLA.

The invention provides a method for detecting a spatial proximity of a first and a second epitope of a protein or of a first and a second protein of a protein complex in a sample of a subject, wherein the method comprises:

binding a first binding member having a first oligonucleotide conjugated thereto to the first epitope,

binding a second binding member having a second oligonucleotide conjugated thereto to the second epitope, and

determining whether a Fluorescence Resonance Energy Transfer (FRET) effect is present between a donor fluorophore and an acceptor fluorophore, which are associated with the first oligonucleotide and the second oligonucleotide, wherein the presence of the FRET effect indicates a spatial proximity of the first and the second oligonucleotide and, thus, the spatial proximity of the first and the second epitope,

wherein the first oligonucleotide is at least partially complementary to the second oligonucleotide,

wherein the first oligonucleotide is initially provided with a first separate shield element and/or the second oligonucleotide is initially provided with a second separate shield element for preventing a premature hybridization of the first and the second oligonucleotide.

Since the first binding member having the first oligonucleotide attached thereto is bound to the first epitope and the second binding member having the second oligonucleotide attached thereto is bound to the second epitope, by determining whether a FRET effect is present between the donor fluorophore and the acceptor fluorophore, wherein the presence of the FRET effect indicates a spatial proximity of the first and the second oligonucleotide, a spatial proximity of the first and the second epitope of the protein or of the first and the second protein of the protein complex can be detected in the sample of the subject. Compared to the standard PLA protocol, embodiments of the present invention may be completed in a shorter time. Moreover, embodiments of the present invention do not require the use of an enzyme or, at least, do not require complex enzymatic reactions, such as sequence amplification or the like.

Since the first oligonucleotide is at least partially complementary to the second oligonucleotide, when in a spatial proximity, the first and the second oligonucleotide will hybridize, whereby the donor fluorophore and the acceptor fluorophore can be brought into a suitable distance from each other, such as to allow a FRET effect to occur between the donor fluorophore and the acceptor fluorophore with a higher certainty. The length of complementary segments should, preferably, be more than 10 nucleotides. Moreover, multiple complementary segments can be present in the oligonucleotides with non-complementary segments in between. The latter may have different lengths for the first and the second oligonucleotide.

Since the first oligonucleotide is initially provided with a first separate shield element and/or the second oligonucleotide is initially provided with a second separate shield element for preventing a premature hybridization of the first and the second oligonucleotide, it is possible to prevent the first and the second oligonucleotide from already hybridizing before the first and the second binding member are bound to the first and the second epitope. Therewith, detection errors, which may be caused, for instance, when the first and the second oligonucleotide would hybridize before the binding allowing for a FRET effect to occur between the donor fluorophore and the acceptor fluorophore and, then, one of the first and the second binding member would bind to the respective epitope of a single protein (or a protein that does not exhibit the other epitope), may be reduced or avoided. Moreover, since the shield element(s) is/are (a) separate shield element(s), i.e., before it is/they are provided to the respective oligonucleotide(s), it is/they are in the form of (a) molecule(s) that is/are separate from the respective oligonucleotide(s), wherein, preferably, it is/they are selected from molecules that are at least partially complementary to the respective oligonucleotide(s) and hybridized thereto, the shielding functionality—as well as the functionality of removing the shielding as described further below—can be provided with a high reliability.

Fluorescence Resonance Energy Transfer (FRET)—also called Förster Resonance Energy Transfer after the German Scientist Theodor Förster—is a mechanism describing energy transfer between two fluorophores, that is, fluorescent chemical compounds that can re-emit light upon light excitation. A donor fluorophore, initially in its electronic excited state, may transfer energy to an acceptor fluorophore through non-radiative dipoledipole coupling. The efficiency of this energy transfer is inversely proportional to the sixth power of the distance between the donor and the acceptor fluorophore, making FRET extremely sensitive to small distances. To enable a FRET effect, part of the emission spectrum of the donor fluorophore has to overlap with the excitation spectrum of the acceptor fluorophore, as it is illustrated in FIG. 1, which schematically and exemplarily shows the spectra of Cy3 and Cy5. As can be seen from the figure, the excitation spectrum of Cy3 (solid curve Ex on the left side) has an excitation maximum at a wavelength of 548 nm and its emission spectrum (stippled curve Em on the left side) is shifted to higher wavelengths with an emission maximum at a wavelength of 562 nm. In contrast, the excitation spectrum of Cy5 (solid curve Ex on the right side) has an excitation maximum at a wavelength of 646 nm and its emission spectrum (stippled curve Em on the right side) is shifted to higher wavelengths with an emission maximum at a wavelength of 664 nm. The shaded region below the emission spectrum of Cy3 and the excitation spectrum of Cy5 indicates the region of overlap required to enable a FRET effect.

FRET is being used in molecular biology (sometimes using genetically encoded fluorescent proteins) to investigate a spatial proximity between two entities, for instance, to explore nucleosome breathing (see Koopmans W. J. et al., “Engineering mononucleosomes for single-pair FRET experiments” Methods in Molecular Biology, Vol. 739, 2011, pages 291 to 303). In fact, FRET is a preferred method to study (biological) interactions on the 1 to 10 nm scale. Popular FRET donor-acceptor fluorophore pairs include: Fluorescein isothiocyanate (FITC)-Tetramethylrhodamine (TRITC), Cy3-Cy5, Enhanced green fluorescent protein (EGFP)-Cy3, Cyan fluorescent protein (CFP)-Yellow fluorescent protein (YFP) and EGFP-YFP.

The presence of a FRET effect can be determined by spectrally analyzing the fluorescence emission in a spatially resolved manner. The dyes of the donor fluorophore and of the acceptor fluorophore are chosen such that the donor fluorophore can be excited without exciting the acceptor fluorophore in the absence of a FRET effect. By exciting the donor fluorophore and measuring the fluorescence emission of the acceptor fluorophore, a detectable signal in the emission channel of the acceptor fluorophore indicates the presence of a FRET effect. FRET efficiency is known to scale with molecular distance, as expressed in the following equation:

$$E = \frac{1}{1 + {( r / {R}_{0} )}^{6}} , \qquad ( 1 )$$

where E designates the FRET efficiency, r designates the molecular distance, and R₀ designates the Förster radius, that is, a parameter that depends on the spectral overlap of the donor fluorophore and the acceptor fluorophore. Typically, it is determined as a ratio between the emission intensities of the donor fluorophore and the acceptor fluorophore—the closer the molecules are, the less emission from the donor fluorophore and the more emission from the acceptor fluorophore is observed.

For the detection of protein interaction on tissue or cytology, fluorescence images can be acquired with excitation in the excitation (absorption) band of the donor fluorophore and detection in the emission band of the acceptor fluorophore. For a check of the FRET efficiency, this can be compared with an image that is acquired with excitation in the excitation (absorption) band of the acceptor fluorophore.

The sample of the subject can be an extracted sample, that is, a sample that has been extracted from the subject. The term “subject”, as used herein, refers to any living being. In some embodiments, the subject is a plant. In some embodiments, the subject is an animal, preferably a mammal. In certain embodiments, the term “subject” refers to a human being, preferably a patient.

Examples of the sample include, but are not limited to, a tissue, cells, blood and/or a body fluid of a subject.

In an embodiment, the first and the second epitopes are two but the same epitopes. In another embodiment, the first and the second epitopes are different from each other.

The first and the second binding member can be a first and a second antibody, preferably, a first and a second monoclonal antibody. Alternatively, however, they can also be, for instance, a first and a second antibody fragment, such as, a camelid antibody, an aptamer or an oligo peptide. More generally, the first and the second binding member can be any kinds of elements or structures that are able to bind to the first and the second epitope, respectively, and to which the first and the second oligonucleotide can be conjugated. In an embodiment, the first and the second binding member are the same. In another embodiment, the first and the second binding member are different from each other.

A suitable length of the first and the second oligonucleotide is 20 to 1000 base pairs, preferably, 30 to 600 base pairs.

In an embodiment, the first and the second oligonucleotide are the same. In another embodiment, the first and the second oligonucleotide are different from each other.

It is noted that while the binding of the first and the second binding member is defined in two steps, this does not mean that these two steps cannot be performed in a different order or, preferably, substantially simultaneously.

Before determining whether a FRET effect is present between a donor fluorophore and an acceptor fluorophore, the donor fluorophore and the acceptor fluorophore have been associated with the first oligonucleotide and the second oligonucleotide.

In some embodiments, the fluorophores can be attached to the oligonucleotides. For example, the oligonucleotides can be pre-labeled with the fluorophores, i.e., the oligonucleotides are labeled with the fluorophores already before the binding of the binding members, preferably, before the binding members having the oligonucleotides conjugated thereto are added to the sample of the subject. In an embodiment, the first oligonucleotide is pre-labeled with the donor fluorophore and/or the second oligonucleotide is pre-labeled with the acceptor fluorophore. Additionally or alternatively, the method further comprises after the binding of the first binding member, attaching the donor fluorophore to the first oligonucleotide, and/or after the binding of the second binding member, attaching the acceptor fluorophore to the second oligonucleotide.

Since the first oligonucleotide is pre-labeled with the donor fluorophore and/or since the second oligonucleotide is pre-labeled with the acceptor fluorophore, and/or since the method further comprises after the binding of the first binding member, attaching the donor fluorophore to the first oligonucleotide, and/or after the binding of the second binding member, attaching the acceptor fluorophore to the second oligonucleotide, the donor fluorophore and the acceptor fluorophore may be brought into a suitable distance from each other such as to allow a FRET effect to occur between the donor fluorophore and the acceptor fluorophore.

It is noted that while the attaching of the donor fluorophore and the acceptor fluorophore to the first and the second oligonucleotide after the binding of the first and the second binding member is defined above in two steps, this does not mean that these two steps cannot be performed in a different order or, preferably, substantially simultaneously.

This allows preventing the first and the second oligonucleotide from already hybridizing before the first and the second binding member are bound to the first and the second epitope. Therewith, detection errors, which may be caused, for instance, when the first and the second oligonucleotide would hybridize before the binding allowing for a FRET effect to occur between the donor fluorophore and the acceptor fluorophore and, then, one of the first and the second binding member would bind to the respective epitope of a single protein (or a protein that does not exhibit the other epitope), may be reduced or avoided.

It is preferred that the method further comprises:

after binding the first binding member, removing the first separate shield element from the first oligonucleotide, and/or

after binding the second binding member, removing the second separate shield element from the second oligonucleotide.

It is noted that while the removing the first and/or the second separate shield element from the first and the second oligonucleotide after the binding of the first and the second binding member is defined above in two steps, this does not mean that these two steps cannot be performed in a different order or, preferably, substantially simultaneously. In particular, it is preferred that the first and/or the second separate shield element are only removed after both the first and the second binding member have bound to their respective epitopes.

The first separate shield element preferably comprises a first DNA or RNA strand that is at least partially complementary to the first oligonucleotide and hybridized thereto and/or the second separate shield element preferably comprises a second DNA or RNA strand that is at least partially complementary to the second oligonucleotide and hybridized thereto.

It is preferred that the removing of the first DNA or RNA strand and/or the second DNA or RNA strand comprises melting the hybridization of the first oligonucleotide and the first DNA or RNA strand and/or the hybridization of the second oligonucleotide and the second DNA or RNA strand.

For instance, in one example, the temperature of the sample is suitably increased in order to achieve the desired melting of the hybridization. In another example, a solvent of the sample is changed in order to perform the melting. After the melting, the unlabeled first and second DNA strand are, preferably, washed away in a suitable further washing step, resulting in substantially only the specifically bound first and second binding members having the first and the second oligonucleotide attached thereto remaining in the sample.

In a preferred variant, the first DNA or RNA strand is a first RNA strand and/or the second DNA or RNA strand is a second RNA strand, wherein the removing of the first RNA strand and/or the second RNA strand comprises a use of an enzyme.

The enzyme can be, for instance, RNase H, which digests the RNA. This variant has the advantage that the entire process becomes isothermal; on the other hand, however, it requires the use of an enzyme.

In another embodiment, the method preferably comprises:

after the binding of the first binding member, providing a third oligonucleotide pre-labeled with the donor fluorophore, wherein the third oligonucleotide is at least partially complementary to the first oligonucleotide and the attaching of the donor fluorophore to the first oligonucleotide comprises hybridizing the third oligonucleotide therewith, and/or

after the binding of the second binding member, providing a fourth oligonucleotide pre-labeled with the acceptor fluorophore, wherein the fourth oligonucleotide is at least partially complementary to the second oligonucleotide and the attaching of the acceptor fluorophore to the second oligonucleotide comprises hybridizing the fourth oligonucleotide therewith.

Here, it is preferable that the first and the second oligonucleotide are partially complementary in order to achieve the desired spatial proximity. The third and the fourth oligonucleotide then preferably hybridize to the first and the second oligonucleotide in-between corresponding complementary segments of the first and the second oligonucleotide.

It is noted that while the providing of the third and the fourth oligonucleotide pre-labeled with the donor fluorophore and the acceptor fluorophore after the binding of the first and the second binding member is defined above in two steps, this does not mean that these two steps cannot be performed in a different order or, preferably, substantially simultaneously.

An advantage of this embodiment is that the oligonucleotides pre-labeled with the donor fluorophore and the acceptor fluorophore can be decoupled from the rest of the detecting elements, which may allow for a simpler testing and switching of fluorophores (for instance, in the case of multiplexing with interfering fluorphore or highly autofluorescent samples). Moreover, it may allow for the production of standardized detecting tests, in particular, when a secondary immuno assay is used and the tests are designed against, for instance, mouse and rat antibody domains.

In a further embodiment, the first oligonucleotide is pre-labeled with the donor fluorophore or the second oligonucleotide is pre-labeled with the acceptor fluorophore, wherein the method comprises:

after the binding of the first and the second binding member, adding the acceptor fluorophore or the donor fluorophore, which intercalates in a double strand formed by a hybridization of the first and the second oligonucleotide.

Here, the donor fluorophore resp. the acceptor fluorophore, which is added only after the binding of the first and the second binding member, is based on an intercalating dye, for instance, DAPI (4′,6-diamidino-2-phenylindole) or YOYO, which is a tetracationic homodimer of Oxazole Yellow. Because intercalating dyes only fluoresce when actually intercalated in double-stranded DNA, it can advantageously be assured that a FRET effect is only caused at the desired location.

The invention also provides a method for detecting a spatial proximity of a first and a second epitope of a protein or of a first and a second protein of a protein complex in a sample of a subject, wherein the method comprises:

binding a first binding member having a first oligonucleotide conjugated thereto to the first epitope,

binding a second binding member having a second oligonucleotide conjugated thereto to the second epitope, and

determining whether a Fluorescence Resonance Energy Transfer (FRET) effect is present between a donor fluorophore and an acceptor fluorophore, which are associated with the first oligonucleotide and the second oligonucleotide, wherein the presence of the FRET effect indicates a spatial proximity of the first and the second oligonucleotide and, thus, the spatial proximity of the first and the second epitope, wherein the method comprises:

after the binding of the first and the second binding member, providing a polymer in which the PI electrons are delocalized along the molecule, wherein the polymer is able to bind to both the first and the second oligonucleotide and to transfer energy from the donor fluorophore to the acceptor fluorophore, or to act as an acceptor and/or a donor to the donor fluorophore and/or the acceptor fluorophore (see Demchenko A. P., “Nanoparticles and nanocomposites for fluorescence sensing and imaging”, Methods and Applications in Fluorescence, Vol. 1, No. 2, 2013).

Since the energy transfer is achieved by means of a third element, that is, the polymer, the first and the second oligonucleotide do not have to be at least partially complementary. This has the advantage that, if the first and the second oligonucleotide are substantially not complementary at all, it is not necessary to provide a shielding of the first and the second oligonucleotide, which can result in a simpler process, since in this case also the step of removing the shielding can be avoided.

It is preferred that for detecting the spatial proximity of the first and the second epitope of the first and the second protein of the protein complex, the first and the second binding member are selected such that the first and the second epitope are not obscured on the first and the second protein of the protein complex.

It is preferred that the determining whether the FRET effect is present comprises:

acquiring at least one fluorescence image of the sample, and

performing a spatially resolved analysis of the at least one fluorescence image for detecting and localizing the FRET effect.

The present invention also provides a method for stratification of a subject suffering from a disease for assessing the suitability of a therapy, wherein the therapy is directed towards a signaling pathway, and/or for prognosis of the outcome of a disease of a subject and/or for prediction and/or detection of therapy resistance of a subject suffering from a disease towards a therapy, wherein the method comprises:

determining the activation status of the signaling pathway by applying the method as defined above for detecting in a sample of the subject whether at least one transcription factor is present.

The method of the invention may be completed in a short time. Moreover, the method does not require the use of an enzyme or, at least, does not require complex enzymatic reactions, such as sequence amplification or the like.

In some embodiments, the disease can be a cancer.

The invention further provides a kit for performing a method as defined by the invention, wherein the kit comprises the following components:

a first binding member having a first oligonucleotide conjugated thereto, wherein the first binding member is directed against a first epitope,

a second binding member having a second oligonucleotide conjugated thereto, wherein the second binding member is directed against a second epitope, and

a donor fluorophore and an acceptor fluorophore,

wherein the first and the second epitope are of a protein or of a first and a second protein of a protein complex,

wherein the first oligonucleotide is at least partially complementary to the second oligonucleotide,

wherein the first oligonucleotide is provided with a first separate shield element and/or the second oligonucleotide is provided with a second separate shield element for preventing a premature hybridization of the first and the second oligonucleotide.

The invention further provides a kit for performing a method as defined by the invention, wherein the kit comprises the following components:

a first binding member having a first oligonucleotide conjugated thereto, wherein the first binding member is directed against a first epitope,

a second binding member having a second oligonucleotide conjugated thereto, wherein the second binding member is directed against a second epitope,

a donor fluorophore and an acceptor fluorophore, and

a polymer in which the PI electrons are delocalized along the molecule, wherein the polymer is able to bind to both the first and the second oligonucleotide and to transfer energy from the donor fluorophore to the acceptor fluorophore, or to act as an acceptor and/or a donor to the donor fluorophore and/or the acceptor fluorophore,

wherein the first and the second epitope are of a protein or of a first and a second protein of a protein complex.

The kits of the invention do not require the use of an enzyme or, at least, does not require agents for complex enzymatic reactions, such as sequence amplification or the like.

In an embodiment, the donor fluorophore and/or the acceptor fluorophore is associated with and/or attached to the first oligonucleotide and/or the second oligonucleotide.

The invention further provides the use of each of the kits of the invention in a corresponding method of the invention.

The use of the kits for the above methods allows the methods to be completed in a short time. Moreover, the use of the kits does not require the use of an enzyme or, at least, does not require complex enzymatic reactions, such as sequence amplification or the like.

The methods and the kits of the invention have the common advantage that they may reduce or avoid detection errors resulting from an undesired occurrence of a FRET effect between the donor fluorophore and the acceptor fluorophore when not both the first binding member and the second binding member are bound to the first and the second epitope of the protein or of the first and the second protein of the protein complex in the sample of the subject, respectively.

It shall be understood that a preferred embodiment of the invention can also be any combination of the dependent claims or above embodiments with the respective independent claim.

These and other aspects of the invention will be apparent from and elucidated with reference to the embodiments described hereinafter.

### BRIEF DESCRIPTION OF THE DRAWINGS

In the following exemplary and schematic drawings:

FIG. 1 shows the excitation spectrum and the emission spectrum of Cy3 and Cy5,

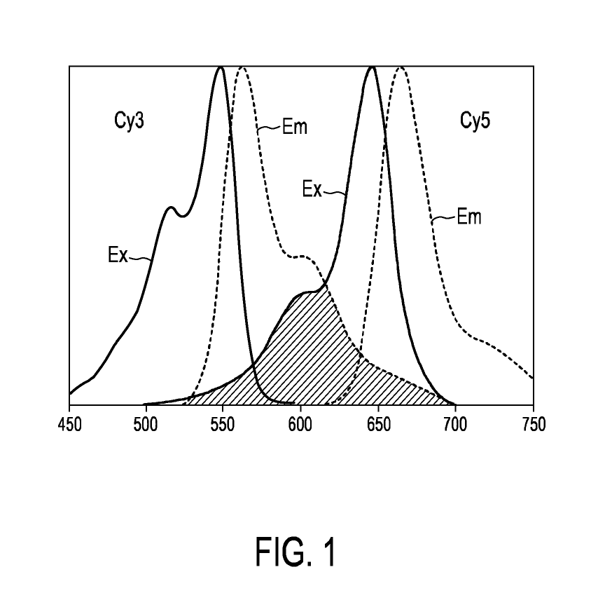

FIG. 2 (a) to (e) shows a first embodiment according to the invention,

FIG. 3 illustrates a variant of the first embodiment shown in FIG. 2 (a) to (e),

FIG. 4 illustrates a second embodiment of a method of the invention,

FIG. 5 illustrates a third embodiment of a method of the invention,

FIG. 6 illustrates a fourth embodiment of a method of the invention.

### DETAILED DESCRIPTION OF EMBODIMENTS

In the figures, like elements are designated with like reference numerals. Moreover, where like elements occur in the same figure or sub-figure, only a single entity may be designated with a reference numeral.

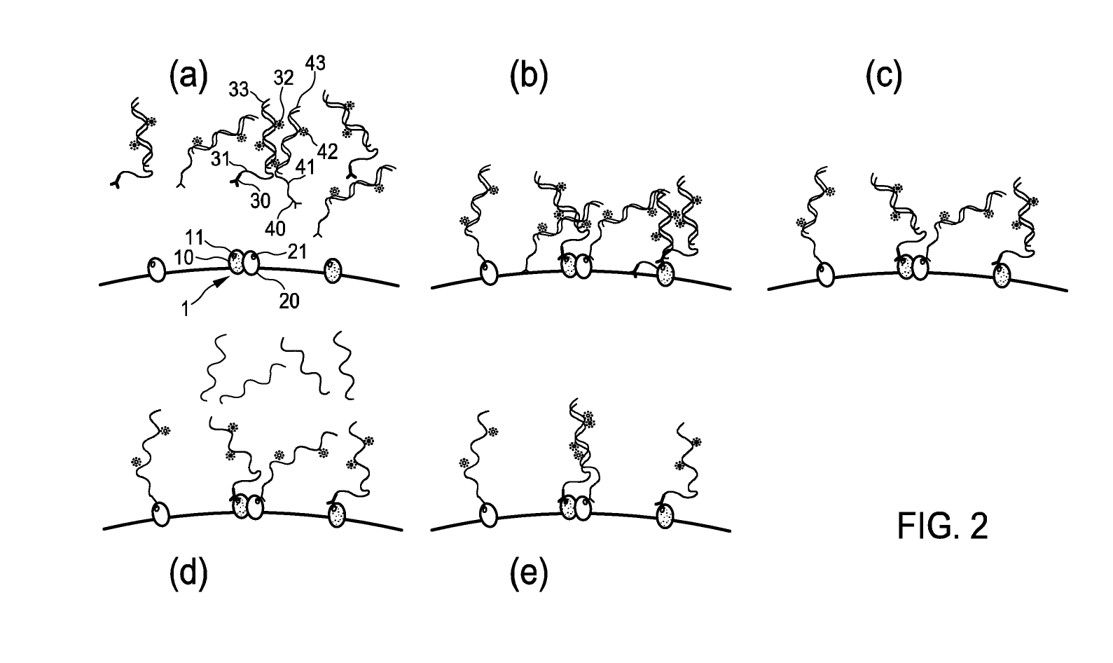

FIG. 2 (a) to (e) shows a first embodiment of a method for detecting a spatial proximity of a first and a second epitope 11, 21 of a first and a second protein 10, 20 of a protein complex 1 in a sample of a tissue and/or cells and/or a body fluid of a patient. As can be seen from the figure, a first binding member 30, here, a first antibody, has a first oligonucleotide 31 conjugated thereto, and a second binding member 40, here, a second antibody, has a second oligonucleotide 41 conjugated thereto.

As shown in FIG. 2 (b), the first antibody 30 having the first oligonucleotide 31 conjugated thereto is bound to the first epitope 11 and the second antibody 40 having the second oligonucleotide 41 conjugated thereto is bound to the second epitope 21. In particular, as shown in FIG. 2 (a), the first antibody 30 having the first oligonucleotide 31 conjugated thereto and the second antibody 40 having the second oligonucleotide 41 conjugated thereto are added to the sample, which comprises the protein complex 1, in this example, a protein dimer consisting of the first and the second protein 10, 20. Additionally, the sample may also comprise the first and the second protein separately, as shown here. Now, after a certain incubation period, the first and the second antibody 30, 40 have bound specifically, that is, have bound to the first and the second epitope 11, 21, or, as the case may be, have bound non-specifically, that is, have bound to locations other than, but possibly similar to, the first and the second epitope 11, 21 (see the antibodies in FIG. 2 (b) that have bound in the space between the illustrated proteins). A suitable washing step is then used to remove possible non-specifically bound first and second antibodies, resulting in substantially only the specifically bound first and second antibodies remaining in the sample, as shown in FIG. 2 (c).

In this embodiment, the first oligonucleotide 31 is pre-labeled with a donor fluorophore 32 and the second oligonucleotide 41 is pre-labeled with an acceptor fluorophore 42. A suitable choice for the donor-acceptor fluorophore pair could be, for instance, Fluorescein isothiocyanate (FITC)-Tetramethylrhodamine (TRITC), Cy3-Cy5, Enhanced green fluorescent protein (EGFP)-Cy3, Cyan fluorescent protein (CFP)-Yellow fluorescent protein (YFP) or EGFP-YFP.

Here, the first and the second oligonucleotide 31, 41 are at least partially complementary, such that they can hybridize when they are in a spatial proximity to each other. In order to prevent a premature hybridization of the first and the second oligonucleotide 31, 41, that is, to prevent the first and the second oligonucleotide 31, 41 from already hybridizing before the first and the second antibody 30, 40 have bound to the first and the second epitope 11, 21, the first oligonucleotide 31 is initially provided with a first separate shield element 33 and the second oligonucleotide 41 is initially provided with a second separate shield element 43. The first and/or the second separate shield element 33, 43 may be much shorter than the first and the second oligonucleotide 31, 41 or it/they may consist of multiple short elements as long as they allow frustrating the hybridization of the first and the second oligonucleotide 31, 41. In this embodiment, the first separate shield element 33 comprises a first DNA strand that is at least partially complementary to the first oligonucleotide 31 and hybridized thereto and the second separate shield element 43 comprises a second DNA strand that is at least partially complementary to the second oligonucleotide 41 and hybridized thereto. The first and the second DNA strand, here, are unlabeled DNA strands, that is, they are not pre-labeled with either the donor fluorophore 32 or the acceptor fluorophore 42.

As shown in FIG. 2 (d), after binding the first antibody 30, the first separate shield element 33, here, the first DNA strand, is removed from the first oligonucleotide 31 and after binding the second antibody 40, the second separate shield element 43, here, the second DNA strand, is removed from the second oligonucleotide 41. In this embodiment, the removing of the first and the second DNA strand comprises melting the hybridization of the first oligonucleotide 31 and the first DNA strand 33 and the hybridization of the second oligonucleotide 41 and the second DNA strand 43. For instance, in one example, the temperature of the sample is suitably increased in order to achieve the desired melting of the hybridization. In another example, a solvent of the sample is changed in order to perform the melting. After the melting, the unlabeled first and second DNA strand 33, 43 are washed away in a suitable further washing step, as also shown in FIG. 2 (d), resulting in substantially only the specifically bound first and second antibodies 30, 40 having the first and the second oligonucleotide 31, 41 attached thereto remaining in the sample.

Once the first and the second separate shield element 33, 43, here, the first and the second DNA strand, have been removed from the first and the second oligonucleotide 31, 41, the two oligonucleotides, which are at least partially complementary, can hybridize, as shown in the center of FIG. 2 (e), bringing the donor fluorophore 32 and the acceptor fluorophore 42 in a spatial proximity that allows for a FRET effect to occur therebetween. (This step preferably comprises lowering the temperature of the sample again after the preceding melting step.) Determining the presence of a FRET effect between the donor fluorophore 32 and the acceptor fluorophore 42, wherein the presence of the FRET effect indicates a spatial proximity of the first and the second oligonucleotide 31, 41, then allows detecting a spatial proximity of a first and a second epitope 11, 21 of the first and the second protein 10, 20 of the protein complex 1 in the sample of the tissue and/or the cells and/or the body fluid of the patient. In contrast, as shown on the left and the right side of FIG. 2 (e), antibodies that have specifically bound to the respective epitope of a single protein will not result in a FRET effect, since the acceptor/donor fluorophore pre-labeled oligonucleotide conjugated thereto will not be in a spatial proximity with an oligonucleotide pre-labeled with a respective donor/acceptor fluorophore.

The first and the second oligonucleotide 31, 41 are preferably pre-labeled on the backbone and not solely at the 3′ or 5′-end as it is usually the case. Moreover, the labels may be applied to specific sites using a labeling as described in Ozaki H. and McLaughlin L. W., “The estimation of distances between specific backbone-labeled sites in DNA using fluorescence resonance energy transfer”, Nucleic Acids Research, Vol. 20, No. 19, 1992, pages 5205 to 5214. It is preferable to also add a base to the sequence that contains a molecule that can be used for site-specific conjugation to antibodies and on top of this there should preferably be a linker of at least 10 to 20 nm (longer in the case where the linker is double-stranded DNA due to the long persistence length) so that the first and the second oligonucleotide 31, 41 may have enough steric freedom to hybridize.

Various ways of achieving efficient energy transfer have been described in the literature (see Demchenko A. P., “Nanoparticles and nanocomposites for fluorescence sensing and imaging”, Methods and Applications in Fluorescence, Vol. 1, No. 2, 2013, 28 pages). Accordingly, it is not necessary to have a symmetric distribution of the donor fluorophore 32 and acceptor fluorophore 42 on the first and the second oligonucleotide 31, 41.

The lower limit of detection (LOD) of a fluorescent scanner or microscope depends very much on the quality of the optics and the camera as well as the conditions of the measurement, such as the integration time and the excitation intensity. A rough estimation is that for a conventional fluorescence microscope optical arrangement about 100 dye molecules per target need to be used assuming having one target in the optical resolution of approximately 0.25 μm². Consequently, in order to detect individual oligonucleotides, an emission intensity that corresponds to about 100 dye molecules is aimed at.

Another design aspect is to avoid homo-FRET interactions between dyes of the same kind. An estimate of what is possible is given on the Invitrogen (Life Technologies/Thermo Fisher) website. According to this, at about 1 dye molecule per 20 base pairs, the Alexa family of dyes is outperforming the traditional cyanine dyes. It has to be noted, however, that this 1:20 density is obtained by random labeling by nick-translation which means that it includes fluorophores less than 20 base pairs apart. By specific labeling higher densities of at least 1 FRET pair per 20 base pairs on each oligonucleotide may be achieved.

Advantageously, an oligonucleotide completely saturated by labels will show homo-FRET, but all donor fluorophore labels may still transfer their energy to their acceptor fluorophore labels, leading to a lower labeling requirement.

Moreover, quenching may also be used to generate an image by subtracting an image obtained before from an image obtained after activation of the FRET-ing oligonucleotides.

Depending on the design and the type of the acceptor fluorophore and the donor fluorophore, for instance, molecule, quantum dot, nano-particle or polymer, the size of the first and the second oligonucleotide 31, 41 may be chosen.

In the case of a classical design with organic fluorescent dyes, the first and the second oligonucleotide 31, 41 should preferably be made quite long to provide enough positions for approximately 100 dye molecules (for instance, 20×100=2000 bases, or 5×100=500 bases).

It is, however, also feasible to chose the number of dyes so small that no individual protein complexes can be detected but rather a certain concentration of complexes such that the total of the emission within the optical resolution of the detector would exceed the detection limit.

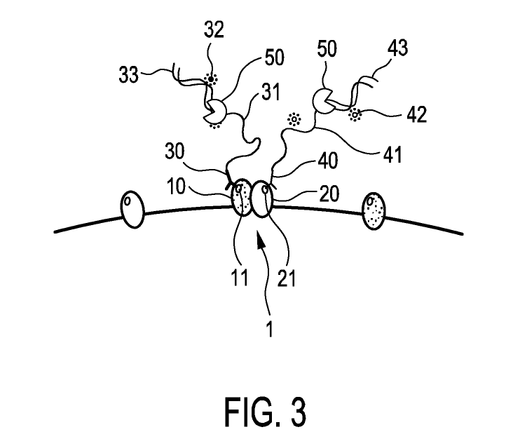

A variant of the first embodiment shown in FIG. 2 (a) to (e) is illustrated in FIG. 3. This variant is substantially similar to the first embodiment. A difference, however, lies in the fact that in this variant, the first separate shield element 33 comprises a first RNA strand that is at least partially complementary to the first oligonucleotide 31 and hybridized thereto and the second separate shield element 43 comprises a second RNA strand that is at least partially complementary to the second oligonucleotide 41 and hybridized thereto. The removing of the first and the second RNA strand then comprises a use of an enzyme 50, for instance, RNase H, for digesting the RNA. This variant has the advantage that the entire process becomes isothermal; on the other hand, however, it requires the use of an enzyme 50.

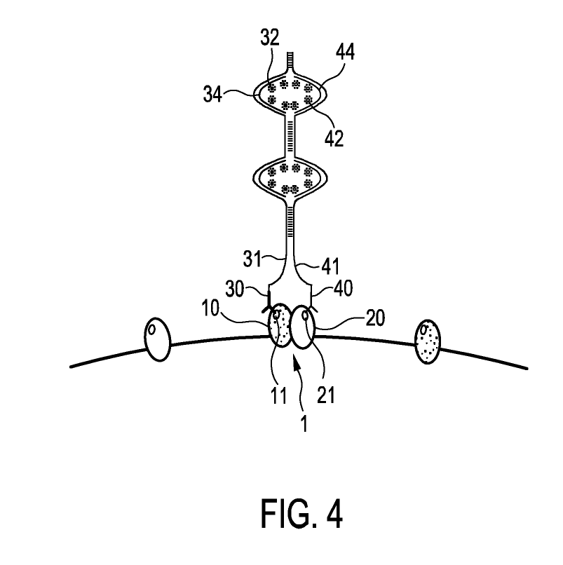

FIG. 4 shows a second embodiment of a method for detecting a spatial proximity of a first and a second epitope 11, 21 of a first and a second protein 10, 20 of a protein complex 1 in a sample of a tissue and/or cells and/or a body fluid of a patient.

This embodiment is substantially similar to the first embodiment shown in FIG. 2 (a) to (e). A difference, however, lies in the fact that in this embodiment, the donor fluorophore 32 is attached to the first oligonucleotide 31 only after the binding of the first binding member 30, here, the first antibody, and the acceptor fluorophore 42 is attached to the second oligonucleotide 41 only after the binding of the second binding member 40, here, the second antibody. In particular, as shown in FIG. 4, after the binding of the first antibody 30, a third oligonucleotide 34 labeled with the donor fluorophore 32 is provided, wherein the third oligonucleotide 34 is at least partially complementary to the first oligonucleotide 31 and the attaching of the donor fluorophore 32 to the first oligonucleotide 31 comprises hybridizing the third oligonucleotide 34 therewith. Likewise, after the binding of the second antibody 40, a fourth oligonucleotide 44 labeled with the acceptor fluorophore 42 is provided, wherein the fourth oligonucleotide 44 is at least partially complementary to the second oligonucleotide 41 and the attaching of the acceptor fluorophore 42 to the second oligonucleotide 41 comprises hybridizing the fourth oligonucleotide 44 therewith.

Here, it is preferable that the first and the second oligonucleotide 31, 41 are partially complementary in order to achieve the desired spatial proximity. The third and the fourth oligonucleotide 34, 44 then preferably hybridize to the first and the second oligonucleotide 31, 41 in-between corresponding complementary segments of the first and the second oligonucleotide 31, 41, as shown in FIG. 4.

An advantage of this embodiment is that the oligonucleotides labeled with the donor fluorophore 32 and the acceptor fluorophore 42 can be decoupled from the rest of the detecting elements, which may allow for a simpler testing and switching of fluorophores (for instance, in the case of multiplexing with interfering fluorophore or highly autofluorescent samples). Moreover, it may allow for the production of standardized detecting tests, in particular, when a secondary immuno assay is used and the tests are designed against, for instance, mouse and rat antibody domains.

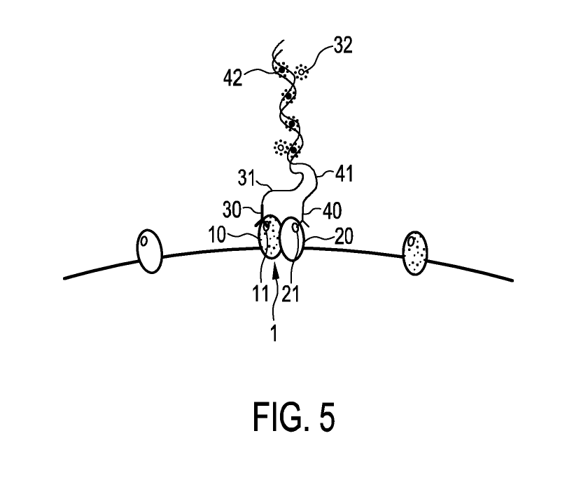

FIG. 5 illustrates a third embodiment of a method for detecting a spatial proximity of a first and a second epitope 11, 21 of a first and a second protein 10, 20 of a protein complex 1 in a sample of a tissue and/or cells and/or a body fluid of a patient.

This embodiment is substantially similar to the first embodiment shown in FIG. 2 (a) to (e). In particular, also in this embodiment, the first and the second oligonucleotide 31, 41 are at least partially complementary, such that they can hybridize when they are in a spatial proximity to each other. A difference, however, lies in the fact that in this embodiment, only the second oligonucleotide 41 is labeled with the acceptor fluorophore 42, and, as shown in FIG. 5, after the binding of the first and the binding member 30, 40, here, the first and the second antibody, the donor fluorophore 32 is added, which intercalates in a double strand formed by a hybridization of the first and the second oligonucleotide 31, 41.

Here, the donor fluorophore 32, which is added only after the binding of the first and the second antibody 30, 40, is based on an intercalating dye, for instance, DAPI (4′,6-diamidino-2-phenylindole) or YOYO, which is a tetracationic homodimer of Oxazole Yellow. Because intercalating dyes only fluoresce when actually intercalated in double stranded DNA, it can advantageously be assured that a FRET effect is only caused at the desired location.

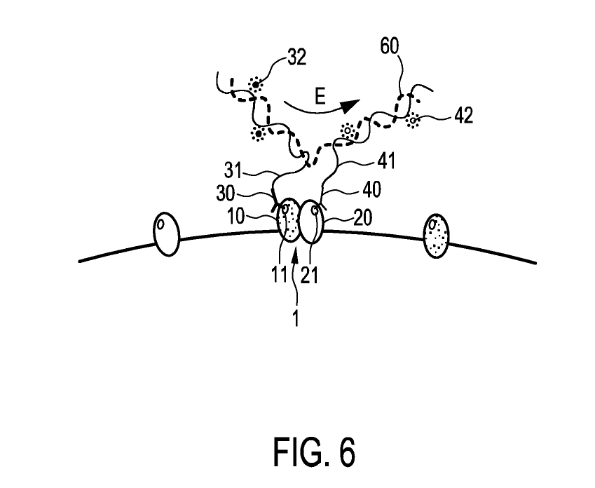

FIG. 6 illustrates a fourth embodiment of a method for detecting a spatial proximity of a first and a second epitope 11, 21 of a first and a second protein 10, 20 of a protein complex 1 in a sample of a tissue and/or cells and/or a body fluid of a patient.

This embodiment is substantially similar to the first embodiment shown in FIG. 2 (a) to (e). A difference, however, lies in the fact that in this embodiment, after the binding of the first and the second binding member 30, 40, here, the first and the second antibody, a polymer 60 in which the PI electrons are delocalized along the molecule is provided, wherein the polymer 60 is able to bind to both the first and the second oligonucleotide 31, 41 and to transfer energy from the donor fluorophore 32 to the acceptor fluorophore 42, or to act as an acceptor and/or a donor to the donor fluorophore 32 and/or the acceptor fluorophore 42.

Since in this embodiment, the energy transfer is achieved by means of a third element, that is, the polymer 60, the first and the second oligonucleotide 31, 41 do not have to be at least partially complementary. This has the advantage that, if the first and the second oligonucleotide 31, 41 are substantially not complementary at all, it is not necessary to provide a shielding of the first and the second oligonucleotide 31, 41, which can result in a simpler process, since in this case also the step of removing the shielding can be avoided.

Preferably, one or more polymers may be linked to one oligonucleotide and one or more quantum dots to the complementary oligonucleotide, leading to very compact detecting elements that can diffuse readily into the sample.

While in the first to fourth embodiment described with reference to FIGS. 2 to 6 above, the first and the second binding member 30, 40 are a first and a second antibody, preferably, a first and a second monoclonal antibody, in other embodiments, they can also be, for instance, a first and a second antibody fragment, such as, a camelid antibody, an aptamer or an oligo peptide. More generally, the first and the second binding member 30, 40 can be any kinds of elements or structures that are able to bind to the first and the second epitope 11, 21, respectively, and to which the first and the second oligonucleotide 31, 41 can be conjugated.

While in the first to fourth embodiment described with reference to FIGS. 2 to 6 above, the binding of the first and the second antibody 30, 40 is defined in two steps, this does not mean that these two steps cannot be performed in a different order or, preferably, substantially simultaneously. The same holds true for the steps of removing the first and the second separate shield elements 33, 43 from the first and the second oligonucleotide 31, 41 and the attaching of the donor fluorophore 32 and the acceptor fluorophore 42 to the first and the second oligonucleotide 31, 41 after the binding of the first and the second antibody 30, 40.

While in the third embodiment described with reference to FIG. 5 above, only the second oligonucleotide 41 is labeled with the acceptor fluorophore 42, and, after the binding of the first and the second antibody 30, 40, the donor fluorophore 32 is added, which intercalates in a double strand formed by a hybridization of the first and the second oligonucleotide 31, 41, it can also be the other way around. In other words: In another embodiment, only the first oligonucleotide 31 can be labeled with the donor fluorophore 32, and, after the binding of the first and the second antibody 30, 40, the acceptor fluorophore 42 can be added, which intercalates in a double strand formed by a hybridization of the first and the second oligonucleotide 31, 41.

In the first to fourth embodiment described with reference to FIGS. 2 to 6 above, the first and the second antibody 30, 40 are preferably selected such that the first and the second epitope 11, 21 are not obscured on the first and the second protein 10, 20 of the protein complex 1.

In the first to fourth embodiment described with reference to FIGS. 2 to 6 above, the present invention has been explained with respect to the detection of a spatial proximity of a first and a second epitope 11, 21 of a first and a second protein 10, 20 of a protein complex 1. However, the present invention can also be employed for the detection of a spatial proximity of a first and a second epitope of a (single) protein.

In the present invention, as described herein, the term “oligonucleotide” is used to include also PNA (peptide nucleic acid) and LNA (locked nucleic acid) molecules.

The use of PNA and/or LNA for the first and/or the second oligonucleotide 31, 41 can be advantageous, since both are known to bind to DNA (and RNA) with a higher specificity. This property may be used, for instance, to make the embodiments in which the first and the second oligonucleotide 31, 41 are at least complementary even more specific and robust by even better preventing a premature hybridization of the first and the second oligonucleotide 31, 41. In addition, if a PNA molecule and/or an LNA molecule is used for the first and/or the second oligonucleotide 31, 41, the temperature increase required for removing the first and the second separate shield element 33, 43 may be lower. Yet further, artificial nucleic acids such as PNA and LNA may have a smaller persistence length.

Additionally, the flexibility of the oligonucleotides may be improved by, for instance, increasing the salt concentration, since in this case, the persistence length decreases consequently. Manning G. S., “The persistence length of DNA is reached from the persistence length of its null isomer through an internal electrostatic stretching force”, Biophysical Journal, Vol. 91, No. 10, 2006, pages 3607 to 3616 indicates that it is possible to bring the persistence length of double-stranded DNA down to 30 nm instead of the normal 50 nm by increasing the salt concentration above 0.1 M.

The present invention can be applied in the field of diagnostics of diseases, in particular, diagnostics of cancer. Examples of multimeric protein aggregates, such as protein dimers, and/or protein posttranslational modifications that could be detected by means of the present invention include:

HER2-HER2 dimers,

HER2-HER3 dimers

HER2 phosphorylation,

AKT phosphorylation,

ER-ER dimers,

ER-p300 dimers,

AR-p300 dimers,

TCF4-β-catenin dimers, etc.

In general, the present invention also relates to a method for stratification of a subject suffering from a disease, preferably a patient, more preferably, a cancer patient, for assessing the suitability of a therapy, wherein the therapy is directed towards a signaling pathway, and/or for prognosis of the outcome of a disease of a subject, preferably cancer of a cancer patient and/or for prediction and/or detection of therapy resistance of a subject suffering from a disease, preferably cancer patient towards a therapy. The method comprises determining the activation status of the signaling pathway by applying a method according to the invention as defined herein above for detecting in a sample of the subject whether at least one transcription factor is present.

Such a method may, for example, be used for detecting the presence of a specific protein, preferably, a transcription factor, such as the membrane receptor HER2, or of two or more spatially proximate proteins, preferably, of two or more proteins being part of a transcription factor complex, such as ER and p300 (see above).

For example, in order to show, in a semi-quantitative fashion, the presence of HER2, immunohistochemistry (IHC) experiments are routinely performed on tissue biopsy samples. The presence or absence of this receptor is clinically relevant as it indicates whether a patient will respond to the targeted drug Herceptin. Other examples of such clinical IHC tests include the detection of the presence of hormone receptors, such as ER and PR, but also of the proliferation marker Ki67, for example.

Although IHC has a proven clinical value, it is limited with respect to the fact that the mere presence of a protein cannot prove its active role in cell signaling. In order to be able to tell whether a protein is actively signaling or not, one needs the method of this application to detect its phosphorylation status or whether it is forming complexes with other proteins. For example, the above mentioned HER2 protein may form dimers with the protein HER3 and circumvent the action of Herceptin. Another example of relevant interactions are transcription factor complexes which are aggregates of multiple proteins whose presence may indicate the activation of gene transcription. Their presence is possibly indicative for a tumor driving pathway and thus relevant for the treatment of said tumor. An example is the transcription factor complex TGF-β/β-catenin: If these proteins can be shown to be in close spatial proximity in the nucleus, the Wnt pathway is most likely in an on-state whereas the mere presence of one of these two proteins alone does not have the same meaning.

As described above, the present invention also relates to a kit for performing a method according to the invention. Depending on the method to be performed, the components of such a kit have to be selected accordingly. For example, for performing a method for assessing the suitability of a Herceptin therapy, such a kit may comprise a first binding member 30, for example, a first antibody, having a first oligonucleotide 31 conjugated thereto, wherein the first binding member 30 is directed against a first epitope 11, a second binding member 40, for example, a second antibody, having a second oligonucleotide 41 conjugated thereto, wherein the second binding member 40 is directed against a second epitope 21, and a donor fluorophore 32 and an acceptor fluorophore 42, wherein the first epitope 11 is of HER2 and the second epitope 21 is of HER3.

Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality.

Any reference signs in the claims should not be construed as limiting the scope.

1. Method for detecting a spatial proximity of a first and a second epitope of a protein or of a first and a second protein of a protein complex in a sample of a subject, wherein the method comprises:
binding a first binding member having a first oligonucleotide conjugated thereto to the first epitope,
binding a second binding member having a second oligonucleotide conjugated thereto to the second epitope, and
determining whether a Fluorescence Resonance Energy Transfer effect is present between a donor fluorophore and an acceptor fluorophore, which are associated with the first oligonucleotide and the second oligonucleotide, wherein the presence of the Fluorescence Resonance Energy Transfer effect indicates a spatial proximity of the first and the second oligonucleotide and, thus, the spatial proximity of the first and the second epitope,
wherein the first oligonucleotide is at least partially complementary to the second oligonucleotide,
wherein the first oligonucleotide is initially provided with a first separate shield element and/or the second oligonucleotide is initially provided with a second separate shield element for preventing a premature hybridization of the first and the second oligonucleotide.

2. The method as defined in claim 1, wherein the first oligonucleotide is pre-labeled with the donor fluorophore and/or wherein the second oligonucleotide is pre-labeled with the acceptor fluorophore, and/or
wherein the method further comprises:
after the binding of the first binding member, attaching the donor fluorophore to the first oligonucleotide, and/or
after the binding of the second binding member, attaching the acceptor fluorophore to the second oligonucleotide.

3. The method as defined in claim 1, wherein the method comprises:
after the binding of the first binding member, removing the first separate shield element from the first oligonucleotide, and/or
after the binding of the second binding member, removing the second separate shield element from the second oligonucleotide.

4. The method as defined in claim 1, wherein the first separate shield element comprises a first DNA or RNA strand that is at least partially complementary to the first oligonucleotide and hybridized thereto and/or the second separate shield element comprises a second DNA or RNA strand that is at least partially complementary to the second oligonucleotide and hybridized thereto.

5. The method as defined in claim 4 when dependent on claim 3, wherein the removing of the first DNA or RNA strand and/or the second DNA or RNA strand comprises melting the hybridization of the first oligonucleotide and the first DNA or RNA strand and/or the hybridization of the second oligonucleotide and the second DNA or RNA strand.

6. The method as defined in claim 3, wherein the first DNA or RNA strand is a first RNA strand and/or the second DNA or RNA strand is a second RNA strand, wherein the removing of the first RNA strand and/or the second RNA strand comprises a use of an enzyme.

7. The method as defined in claim 2, wherein the method comprises:
after the binding of the first binding member, providing a third oligonucleotide pre-labeled with the donor fluorophore, wherein the third oligonucleotide is at least partially complementary to the first oligonucleotide and the attaching of the donor fluorophore to the first oligonucleotide comprises hybridizing the third oligonucleotide therewith, and/or
after the binding of the second binding member, providing a fourth oligonucleotide pre-labeled with the acceptor fluorophore, wherein the fourth oligonucleotide is at least partially complementary to the second oligonucleotide and the attaching of the acceptor fluorophore to the second oligonucleotide comprises hybridizing the fourth oligonucleotide therewith.

8. The method as defined in claim 1, wherein the first oligonucleotide is pre-labeled with the donor fluorophore or the second oligonucleotide is pre-labeled with the acceptor fluorophore, wherein the method comprises:
after the binding of the first and the second binding member, adding the acceptor fluorophore or the donor fluorophore, which intercalates in a double strand formed by a hybridization of the first and the second oligonucleotide.

9. Method for detecting a spatial proximity of a first and a second epitope of a protein or of a first and a second protein of a protein complex in a sample of a subject, wherein the method comprises:
binding a first binding member having a first oligonucleotide conjugated thereto to the first epitope,
binding a second binding member having a second oligonucleotide conjugated thereto to the second epitope, and
determining whether a Fluorescence Resonance Energy Transfer effect is present between a donor fluorophore and an acceptor fluorophore, which are associated with the first oligonucleotide and the second oligonucleotide, wherein the presence of the Fluorescence Resonance Energy Transfer effect indicates a spatial proximity of the first and the second oligonucleotide and, thus, the spatial proximity of the first and the second epitope, wherein the method comprises:
after the binding of the first and the second binding member, providing a polymer in which the PI electrons are delocalized along the molecule, wherein the polymer is able to bind to both the first and the second oligonucleotide and to transfer energy from the donor fluorophore to the acceptor fluorophore.

10. The method as defined in claim 1, wherein the determining whether the Fluorescence Resonance Energy Transfer effect is present comprises:
acquiring at least one fluorescence image of the sample, and
performing a spatially resolved analysis of the at least one fluorescence image for detecting and localizing the Fluorescence Resonance Energy Transfer effect.

11. A method for stratification of a subject suffering from a disease for assessing the suitability of a therapy, the therapy being directed towards a signaling pathway, and/or for prognosis of the outcome of a disease of a subject and/or for prediction and/or detection of therapy resistance of a subject suffering from a disease towards a therapy, wherein the method comprises:
determining the activation status of the signaling pathway by applying the method as defined in claim 10 for detecting in a sample of the subject whether at least one transcription factor is present.

12. A kit for performing the method as defined in claim 1, wherein the kit comprises the following components:
a first binding member having a first oligonucleotide conjugated thereto, wherein the first binding member is directed against a first epitope,
a second binding member having a second oligonucleotide conjugated thereto, wherein the second binding member is directed against a second epitope, and
a donor fluorophore and an acceptor fluorophore,
wherein the first and the second epitope are of a protein or of a first and a second protein of a protein complex,
wherein the first oligonucleotide is at least partially complementary to the second oligonucleotide,
wherein the first oligonucleotide is provided with a first separate shield element and/or the second oligonucleotide is provided with a second separate shield element for preventing a premature hybridization of the first and the second oligonucleotide.

13. A kit for performing the method as defined in claim 11, wherein the kit comprises the following components:
a first binding member having a first oligonucleotide conjugated thereto, wherein the first binding member is directed against a first epitope,
a second binding member having a second oligonucleotide conjugated thereto, wherein the second binding member is directed against a second epitope,
a donor fluorophore an acceptor fluorophore, and
a polymer in which the PI electrons are delocalized along the molecule, wherein the polymer is able to bind to both the first and the second oligonucleotide and to transfer energy from the donor fluorophore to the acceptor fluorophore,
wherein the first and the second epitope are of a protein or of a first and a second protein of a protein complex.

14. (canceled)
